(19) United States
(12) Patent Application Publication
(10) Pub. No.: US 2015/0306377 A1
(43) Pub. Date: Oct. 29, 2015

(54) PORTABLE CARDIAC DEFIBRILLATORS WITH SUBCUTANEOUS ELECTRODES AND ASSOCIATED SYSTEMS AND METHODS

(71) Applicant: John W. Brantigan, Shaw Island, WA (US)

(72) Inventor: John W. Brantigan, Shaw Island, WA (US)

(21) Appl. No.: 14/530,470

(22) Filed: Oct. 31, 2014

Related U.S. Application Data
(60) Provisional application No. 61/985,359, filed on Apr. 28, 2014.

(51) Int. Cl. A61N 1/05; A61N 1/39; A61N 1/08

(52) U.S. Cl. CPC A61N 1/0504; A61N 1/08; A61N 1/3987; A61N 1/3993

(57) ABSTRACT

The present disclosure is directed to portable cardiac defibrillators with subcutaneous electrodes and associated systems and methods. In one embodiment, a portable external cardiac defibrillator system for treating a human patient can include a first electrode, a second electrode, and an electrical pulse generator external to the human patient and operably coupled to the first and second electrodes. The first and second electrodes can be configured to be external to the human patient and implanted subcutaneously in the human patient during a cardiac emergency. The electrical pulse generator can be configured to deliver an electrical shock to the human patient via the first and second electrodes while the first and second electrodes are subcutaneously implanted to provide internal defibrillation to the human patient.

### CROSS-REFERENCE TO RELATED APPLICATION

The present application claims priority to U.S. Provisional Patent Application No. 61/985,359, filed Apr. 28, 2014, which is incorporated by reference herein in its entirety.

### TECHNICAL FIELD

The present technology relates generally to cardiac defibrillators. In particular, at least some embodiments in accordance with the present technology are related to cardiac defibrillators with subcutaneous electrodes.

### BACKGROUND

Common causes of sudden cardiac death include ventricular fibrillation (“V-fib” or “VF”), cardiac dysrhythmias, and pulseless ventricular tachycardia. During VF, the heart's electrical activity suddenly becomes disordered and causes the heart's ventricles to flutter in a rapid, disorganized pattern. This uncoordinated contraction of the cardiac muscles pumps little to no blood through the body, thereby causing the affected individual to collapse and go into sudden cardiac arrest. Irreversible brain death typically occurs within a few minutes.

Electrical defibrillation can counter such arrhythmic heart conditions (e.g., VF). The electrical energy delivered by a cardiac defibrillator can terminate the disorganized rhythm of the heart and allow normal sinus rhythm to be re-established by the sinoatrial node of the heart (i.e., the body's natural pacemaker). The survival rate during VF is thought to be directly related to the time interval between the onset of VF and the delivery of a therapeutic dose of electrical energy to the heart with the defibrillator.

Early defibrillators used paddle-type electrodes that were applied directly to the heart to deliver alternating current of up to 1000 V. These devices were typically large and bulky, needed to be plugged into a wall outlet, and often caused muscle damage to the heart. Eventually, external defibrillators were developed and widely adopted for hospital use. These units traditionally produced monophasic electrical waveforms, delivering a first shock of 200 J, followed by second and third shocks (as required) with energy levels rising as high as 360 J. The resistance to current flow caused by delivering energy through a patient's skin (i.e., transthoracic impedance) required external defibrillators to deliver large amounts of electrical energy. To reduce the impedance, external defibrillators included large surface-area electrode paddles and used a gel disposed between the paddles and the skin to improve contact. However, problems still arose with the large external paddles. For example, hirsute patients often caused air trapping, a high impedance, and occasional “arcing,” which resulted in burns.

Portable external defibrillators are widely used, but they also have drawbacks. For example, they require large, heavy batteries to provide sufficient energy outputs for the defibrillator, and these batteries require regular maintenance and re-charging. Thus, it was not uncommon for defibrillation to fail because a portable external defibrillator had not been properly maintained.

In the 1970's to 1980's, several research groups developed fully implantable cardiac defibrillators (“ICDs”) that could automatically detect VF and ventricular tachycardia (“VT”), and deliver electrical shocks to the cardiac muscles (see, e.g., U.S. Pat. No. 4,765,341, which is incorporated herein by reference in its entirety). The electrodes of the ICDs were sutured to the heart and, therefore, implantation of ICDs required a thoracotomy. Due to the highly invasive procedure, ICDs were typically reserved for patients who had survived at least one prior cardiac arrest and had a high probably of reoccurrence.

Further development of ICDs provided transvenous insertion of electrodes (e.g., as disclosed in U.S. Pat. No. 4,603,705, U.S. Pat. No. 4,944,300, and U.S. Pat. No. 5,105,810, each of which is incorporated herein by reference in its entirety), and eliminated the need for major surgery. These devices became more widely used and have saved many lives. However, while minimally invasive, the transvenously-implanted ICDs created risks to the heart, such as perforation, pericardial effusion, cardiac tamponade, and/or valve damage. For example, because the ICDs were tranvenously implanted and located in a patient's vasculature, any infection surrounding the ICDs affected the patient's entire body and could be life-threatening. In addition, the electrodes of a transvenously-implanted ICD were exposed to cardiac contractions, and therefore subject to repetitive bending, lead fracture, and/or failure. Removal of broken electrode leads resulted in vessel perforation, occlusion, bleeding, and/or death.

Automated external defibrillators (“AEDs”) have also been developed that analyze heart rhythm and advise as to whether a shock should be delivered. These devices are designed to be used by lay persons with little or no medical training. AEDs have been widely adopted and placed in locations where large numbers of people routinely congregate, such as airports, shopping centers, and government buildings. AEDs are often brightly colored to attract attention, and are typically wall-mounted in prominent locations. One drawback of AEDs is that they are too large and heavy to be manually carried for long periods. Another drawback is that the automated heart rhythm analysis performed by AEDs can take valuable time, typically about 20 seconds, during which regular chest compressions of cardio pulmonary resuscitation (“CPR”) must be halted. This delay can impair the success of defibrillation. Thus, AEDs should not be used when trained professionals have ready access to manual defibrillators that diagnose and treat arrhythmias faster.

An entirely subcutaneous implanted cardiac defibrillator (“S-IDC”) is disclosed in U.S. Pat. No. 8,577,454, which is incorporated herein by reference in its entirety. The S-ICD includes an electrical lead with a proximal sensing electrode, a distal sensing electrode, and an intermediate defibrillation coil. The device is placed entirely subcutaneously such that the electrical lead extends from the left side of the upper sternum to approximately the mid-axillary line between the fifth and sixth intercostal spaces where an electrical pulse generator is implanted in a subcutaneous pocket. The 5-ICD delivers shocks of up to 80 J, which is approximately half the output of external devices. This device avoids the complications associated with ICDs with transvenously placed electrodes. For example, any infections resulting from the placement of the S-IDC would be cutaneous, and therefore much less dangerous than intravenous infections and much easier to treat. However, S-IDCs are best suited for patients with a history of cardiac arrhythmias because they are permanently implanted into the patient. Therefore, S-IDCs do not benefit those who suffer from unexpected cardiac arrhythmias and/or those not suited for the implantation of S-IDCs.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology, For ease of reference, through this disclosure identical reference numbers may be used to identify identical or at least generally similar or analogous components or features.

FIG. 1 is an isometric view of a portable, partially subcutaneous cardiac defibrillator system configured in accordance with an embodiment of the present technology.

FIG. 2 is an enlarged side view of electrodes of the portable, partially subcutaneous cardiac defibrillator system of FIG. 1 configured in accordance with an embodiment of the present technology.

FIG. 3 is an isometric view of the portable, partially subcutaneous cardiac defibrillator system of FIG. 1 having a compartment configured in accordance with an embodiment of the present technology.

FIG. 4 is a partially schematic view illustrating a portable partially subcutaneous cardiac defibrillator system implanted in a human patient in accordance with an embodiment of the present technology.

### DETAILED DESCRIPTION

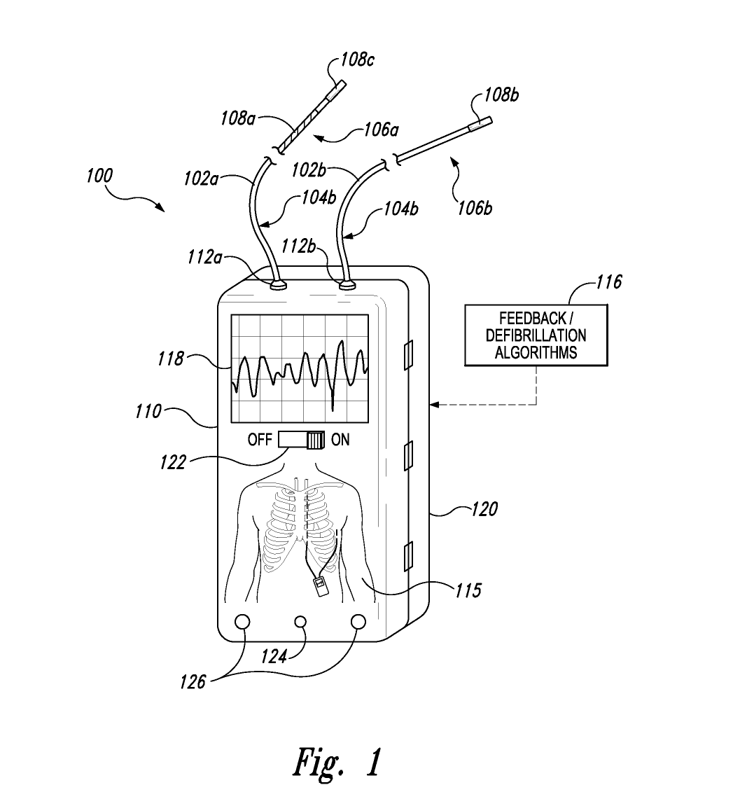

Portable external cardiac defibrillators with subcutaneous electrodes and associated systems and methods are disclosed herein. Cardiac defibrillator systems configured in accordance with at least some embodiments of the present technology can include an external electrical pulse generator capable of being manually activated to deliver electrical shock to achieve internal cardiac defibrillation using subcutaneously positioned electrodes. In various embodiments, the cardiac defibrillator system can be easily carried by a user. For example, the cardiac defibrillator system can be carried in the pocket of a user, on a holster, and/or other suitable transport mechanism. As used herein, the term “user” can refer to a physician and/or an individual trained to use the portable cardiac defibrillator. Several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described below with reference to FIGS. 1-4.

As used herein, the terms “distal” and “proximal” define a position or direction with respect to a user and/or an externally located electrical pulse generator. The terms, “distal” and “distally” refer to a position distant from or in a direction away from the user or the pulse generator. The terms “proximal” and “proximally” refer to a position near or in a direction toward the user or the pulse generator.

FIG. 1 is an isometric view of a portable, partially subcutaneous cardiac defibrillator system 100 (“defibrillator system 100”) configured in accordance with an embodiment of the present technology. The defibrillator system 100 includes at least two electrical leads 102 (identified individually as a first or anterior lead 102a and a second or lateral lead 102b; also referred to as “percutaneous leads 102”), and each lead 102 has a proximal portion 104 (identified individually as a first proximal portion 104a and a second proximal portion 104b) and a distal portion 106 (identified individually as a first distal portion 106a and a second distal portion 106b; also referred to as “active portions 106”). The distal portions 106 of the two leads 102 can each include at least one electrode 108 (identified individually as a first electrode 108a of the first lead 102a and a second electrode 108b of the second lead 102b). The defibrillator system 100 can further include an electrical pulse generator module 110 that is electrically coupled to the first and second electrodes 108a and 108b via the corresponding first and second electrical leads 102a and 102b. For example, as illustrated in FIG. 1, the proximal portions 104 of the first and second electrical leads 102a and 102b are coupled to the pulse generator module 110 via two corresponding receptacles or ports 112 (identified individually as a first port 112a for connection to the first lead 102a and a second port 112b for connection to the second lead 102b). In other embodiments, the leads 102 can be electrically coupled to the pulse generator module 110 using other suitable means (e.g., a single port that is split into two separate electrical channels).

In a cardiac emergency (e.g., during VF), the distal portions 106 of the two leads 102 can be subcutaneously inserted into a human patient and placed across a portion of the patient's heart. In certain embodiments, for example, the distal portion 106a of the first lead 102a is subcutaneously positioned to the left of the upper sternum of the patient, and the distal portion 106b of the second lead 102b is subcutaneously positioned at the mid-axillary line between intercostal spaces of the patient (e.g., between the fifth and sixth intercostal spaces, between the fourth and fifth intercostal spaces, etc.). In other embodiments, the distal portions 106 of the leads 102 can be positioned elsewhere around the heart suitable for electrical defibrillation or cardioversion. The proximal portions 104 of the leads 102 extend outside the human patient to where they connect with the pulse generator module 110 via the corresponding ports 112. The pulse generator module 110 can be activated to deliver one or more therapeutic doses of electrical energy to the heart via the electrodes 108 at the distal portions 106 of the electrical leads 102.

The leads 102 can include one or more electrical wires and/or other conductive elements that can connect the electrodes 108 to the pulse generator module 110. The leads 102 may be encapsulated and/or otherwise covered by an electrically insulating material to protect and electrically insulate the leads 102 from the environment, such as silicone or polyurethane insulation. At least a portion of the insulating material housing the leads 102 can be made from a biocompatible material that is suitable for being subcutaneously implanted into a human patient for at least a period of time long enough to deliver pulses of electrical energy for cardioversion. As described in further detail below, in certain embodiments more than one electrode 108 and/or other electronic element (e.g., a sensor) may be located at the distal portions 106 of the leads 102. In these embodiments, the first and/or second lead 102 can include an insulating material that houses multiple electrical connecting wires or leads, and each wire can be connected independently to a different electrode 102 and/or electronic element at the distal portion 106 of the corresponding lead 102.

In various embodiments, the distal portions 106 of the leads 102 can be moderately stiff and configured specifically to locate the electrodes 108 in their respective target sites. For example, the distal portion 106a of the first lead 102a can be configured to allow the first electrode 108a to be threaded subcutaneously from the xiphoid process of a patient to the left of the patient's upper sternum. Similarly, the distal portion 106b of the second lead 102b can be configured to allow the second electrode 108b to be threaded subcutaneously from the patient's mid-axillary line of the human patient to the intercostal spaces (e.g., the fifth and sixth intercostal spaces). In other embodiments, the distal portions 106 of the leads 106 can be configured to subcutaneously locate the electrodes 108 at different target sites.

In certain embodiments, the ports 112 and the leads 102 can be color-coded to visually indicate to the user which lead 102 should be connected to which port 112. For example, the first lead 102a and the first port 112a may have a first color (e.g., black), and the second lead 102b and the second port 112b may have a second color (e.g., red) different from the first color. In other embodiments, the leads 102 and the ports 112 can have other indicators that correspond to one another to facilitate connection of the leads 102 to the appropriate ports 112 on the pulse generator module 110, or the first lead 102a and first port 112a can have a first size and/or shape and the second lead 102b and second port 112b can have a second size and/or shape different than the first size/shape. In further embodiments, the leads 102 may be preinstalled in the ports 112 or otherwise permanently connected to the pulse generator module 110.

The pulse generator module 110 can deliver electrical shocks in biphasic wave forms at energy levels sufficient to subcutaneously provide cardiac defibrillation. The pulse generator module 110 can include a source of electrical energy (e.g., a battery supply), at least one capacitor, and, optionally, operational circuitry. The pulse generator module 110 typically delivers energy and voltage levels greater than completely implanted cardiac defibrillators (e.g., 750 V and 40 J) that directly contact the heart, but much less than external defibrillators (e.g., 2,000-3,000 V and 150-360 J) because the electrodes 108 are implanted subcutaneously and therefore bypass the impedance or resistance of the skin. As such, the pulse module generator 110 can be configured to have a maximum of energy of 40-100 J and voltages in the range of 800-2,000 V. In certain embodiments, for example, the pulse module generator 110 has a maximum voltage of 80 J or less. The lower levels of energy required for subcutaneous defibrillation enable the pulse generator module 110 to be relatively small in comparison to external defibrillator systems. For example, the pulse generator module 110 may have similar dimensions and/or weight as a standard smart phone (e.g., approximately 120-160 mm by 50-80 mm by 6-10 mm; weighing less than 200 grams). This allows the user to easily carry the pulse generator module 110 in his or her pocket, on a holster, and/or otherwise on the user, making the defibrillator system 100 highly portable and easily accessible for quick availability during cardiac emergencies.

As further shown in FIG. 1, the pulse generator module 110 can include a housing 120 that at least partially encases the circuitry of the pulse generator module 110. The housing 120 can be made from a durable material, such as plastic. The pulse generator module 110 can further include an on-off switch or button 122 on the exterior of the housing 120 that activates the pulse generator module 110 and begins charging the capacitors. When the capacitors are sufficiently charged, an indicator light 124 can illuminate to notify to the user that the pulse generator module 110 is ready to deliver defibrillation energy. In other embodiments, the pulse generator module 110 can include other types of indicators, such as audible signals (e.g., a beep or verbal notification), to indicate that the defibrillation device is ready for use. In further embodiments, the on-off switch 122 and/or the indicator light 124 can be positioned on different portions of the housing 120 or the defibrillator system 100. For example, in various embodiments the pulse generator 110 module may be activated by a remote.

The pulse generator module 110 can further include an activation mechanism 126 that can be manually manipulated by the user to administer the defibrillation shock. In the embodiment illustrated in FIG. 1, for example, the activation mechanism 126 is defined by two buttons that must be compressed simultaneously to initiate defibrillation. This embodiment is expected to reduce the likelihood of accidental discharge that may occur when inadvertently pushing a single button. In other embodiments, the activation mechanism may include a lever, a switch, a single button, a fingerprint scanner, and/or other suitable mechanisms that can activate energy delivery.

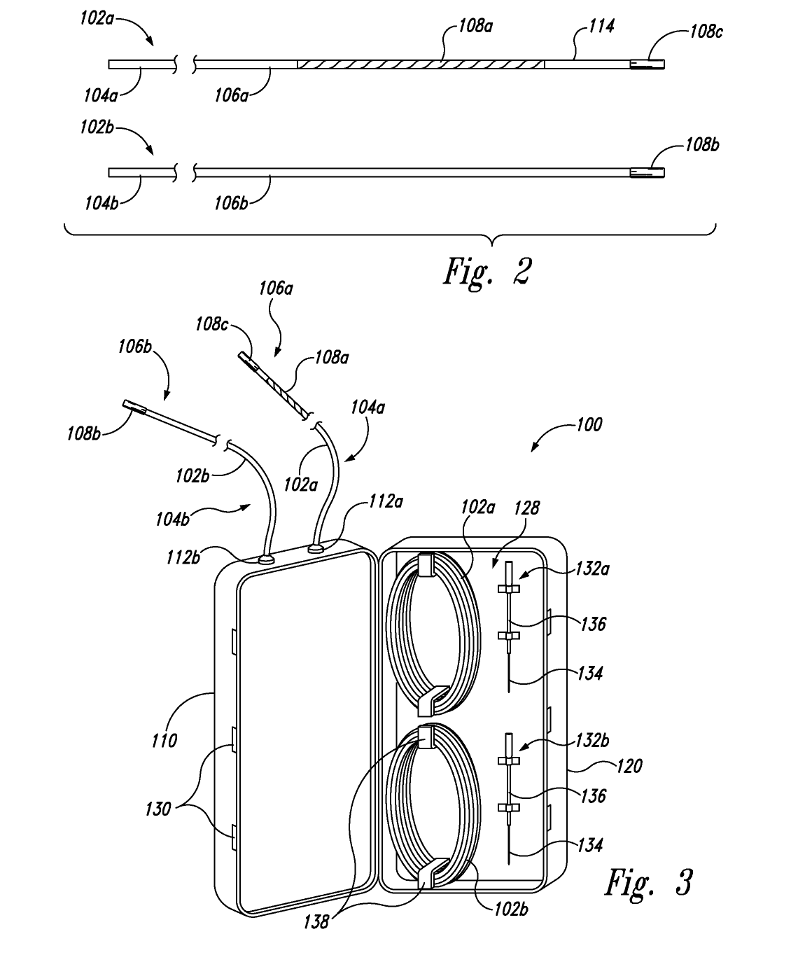

FIG. 2 is an enlarged side view of the distal portions 106 of the leads 102. As shown in FIG. 2, the first distal portion 106a includes the first electrode 108a, and the second distal portion 106b includes the second electrode 108b such that the two electrodes 108 can electrically communicate with each other to deliver electrical energy across the electrodes 108 and, when implanted in a human patient, across the heart of the patient. For example, the first electrode 108a can serve as a cathode and the second electrode 108b can serve as an anode, or vice versa. In the illustrated embodiment, the first electrode 108a is defined by a defibrillation coil extending along a section of the first lead 102a, and the second electrode 108b is defined by a sensing electrode at the distal end of the second lead 102b. The electrodes 108 can be made from various electrically conductive materials, such as an aluminum alloy, stainless steel, platinum, platinum iridium alloys, and other electrically conductive biocompatible materials. In certain embodiments, the first electrode 108a (i.e., the defibrillation coil) is about 5-10 cm (e.g., 8 cm) in length, and the overall length of the first lead 102a is about 20-50 cm (e.g., 25 cm, 30 cm, 32 cm, etc.). The second electrode 108b (i.e., the sensing electrode) can be defined by an end portion of the second lead 102b, and the second lead 102b can have an overall length of about 10-30 cm (e.g., 15 cm, 17 cm, 23 cm, etc.). This configuration allows at least 10 cm of the first lead 102a and at least 5 cm of the second lead 102b to be threaded under the skin of the patient. In other embodiments, the leads 102 and/or the electrodes 108 can have different lengths, the first electrode 108a and/or the second electrode 108b can be positioned in other locations along the distal portions 106 of the leads 102, and/or the electrodes 108 can be made from other suitable types of electrodes.

As shown in FIG. 2, the first lead 102a can further include a third electrode 108c that is spaced apart from the first electrode 108a by an insulated portion 114. The insulated portion 114 can be made from a dielectric material, such as silicone or a polyurethane material. The first lead 102a can include two conductors (e.g., wires) that extend along its length, with one wire electrically coupled to the first electrode 108a and the other wire electrically coupled to the third electrode 108c independently of the first electrode 108a. In the illustrated embodiment, the third electrode 108c is at the distal end of the first distal portion 106a, but in other embodiments the third electrode 108c can be spaced along the length of the first distal portion 106a distally or proximally to the first electrode 108a and electrically isolated therefrom. For example, the third electrode 108c can extend around the circumference of the first lead 102a or a portion thereof.

The third electrode 108c can define a sense electrode that can operate in conjunction with the second electrode 108b of the second lead 102b to detect cardiac rhythms, such as QRS waves typically recorded via an electrocardiogram (“ECG”). The cardiac rhythms detected by the second and third electrodes 108b and 108c (also referred to as “sense electrodes”) can be communicated to the pulse generator module 110 (FIG. 1) via the leads 102. In other embodiments, the sensing of QRS waves can be performed by a combination of the defibrillation coil of the first electrode 108a and the second electrode 108b and/or other electrode configurations. For example, in certain embodiments, one of the leads 102 can include two sensing electrodes spaced apart from one another and configured to detect cardiac rhythms.

Referring back to FIG. 1, the pulse generator module 110 can include feedback/defibrillation algorithms 116 (shown schematically) that process and/or monitor the cardiac signals detected by the electrodes 108 (e.g., the second and third electrodes 108b and 108c). In various embodiments, the cardiac rhythms (e.g., QRS waveforms) processed by the algorithms 116 can be displayed to a user in real time on a display 118, such as an LCD screen or other suitable monitor. In certain embodiments. For example, when the user is a physician or other person skilled at reading electrocardiograms, the display 118 can be used to determine the correct time for applying an electrical pulse to the heart of the patient. In other embodiments, the algorithms 116 can be configured to monitor cardiac activity and automatically initiate charging of the capacitor of the pulse generator module 110 and/or delivery of defibrillation energy via the first and second electrodes 108a and 108b. In further embodiments, the algorithms 116 can monitor cardiac activity and indicate to the user via the display 118 and/or other suitable indicator (e.g. a light) when to apply the defibrillation energy.

In still further embodiments, the pulse generator module 110 can be configured to operate under two different modes: a manual mode and an automatic mode. In the manual mode, the algorithm 116 can include electrical analysis functions that display QRS waveforms to the user via the display 118, and the user can decide when to manually administer the electrical shocks based on this information. This mode may provide for faster application of electrical pulses than an automatic mode because an algorithm does not need to be run before the electrical pulse is applied. It provides the user with freedom to apply the electrical pulse when desired, which may be preferable for a physician who can use his or her knowledge to determine when to initiate energy delivery. When the pulse generator module 110 is in the automatic mode, the algorithm 116 can implement instructions for electrical analysis to either advise the user when to deliver a shock and/or automatically deliver defibrillatory shocks when the algorithm 116 detects the requisite arrhythmia. This fully-automatic mode allows the defibrillator system 100 to stay partially subcutaneously implanted in the patient after initial defibrillation, and automatically provide any necessary shocks until the patient is able to receive further treatment. For example, the fully automatic mode may be used after initial defibrillation to provide further treatment until the patient reaches a hospital for definitive care.

As shown in FIG. 1, the pulse generator module 110 can further include an illustration 115 or other type of display that indicates predetermined points of insertion for the leads 102 and/or predetermined locations for the electrodes 108 with respect to the human body to aid the user in placing the leads 102 in the desired position for delivering defibrillation energy. As shown by the illustration 115, for example, the anterior or first lead 102a can be inserted just to the patient's left of the xiphoid process and threaded under the patient's skin such that the first electrode 108a is positioned to the left side of the patient's upper sternum. The illustration 115 further shows that the lateral or second lead 102b can be inserted proximate to the patient's mid-axillary line and threaded under the patient's skin such that the second electrode 108b is positioned approximately at the mid-axillary line between the fifth and sixth intercostal spaces. In other embodiments, the illustration 115 can provide alternative positions for the leads 102 and/or insertion points. For example, in other embodiments the first electrode 108a can be positioned in the left mid-clavicular line at about the fifth rib and/or the second electrode 108b can be positioned at the posterior axillary line, lateral to the left scapula.

FIG. 3 is an isometric view of the defibrillator system 100 of FIG. 1 illustrating an embodiment in which the housing 120 further includes an interior compartment 128. In the illustrated embodiment, the compartment 128 can be accessed by manipulating a plurality of fasteners 130 along an exterior edge of the housing 120. In other embodiments, the compartment 128 may be accessed using other suitable release mechanisms, such as buttons and levers. As shown in FIG. 3, the compartment 128 can be sized and shaped to store the electrical leads 102 when not in use (i.e., plugged into the ports 112). For example, in the illustrated embodiment the interior of the housing 120 that defines the compartment 128 includes a plurality of hooks 138 around which the leads 102 are wrapped. In other embodiments, the interior of the housing 120 can include other fasteners to secure the leads 102 and/or other components of the system 100 in place within the compartment 128.

As further shown in FIG. 3, the compartment 128 can also be sized to store a plurality of introducers (identified individually as a first introducer 132a and a second introducer 132b; referred to collectively as “introducers 132”) that facilitate insertion of the distal portions 106 of the leads 102 into a patient's subcutaneous space. For example, in certain embodiments each introducer 132 includes an insertion needle 134 with an outer sleeve 136 over at least a portion of the needle 134. The needles 134 can be made from stainless steel and/or other suitable materials sufficiently strong enough to pierce through human skin, and the sleeves can be made from plastic and/or other suitable materials for percutaneous delivery of the leads 102. In various embodiments, the needles 134 can have different lengths depending upon the desired insertion point. For example, the needle 134 used in conjunction with the anterior/first electrical lead 102a can have a length of about 10 cm, and the needle 134 used in conjunction with the lateral/second electrical lead 102b can have a length of about 5 cm. In other embodiments, the needles 134 may have other suitable lengths for subcutaneous lead delivery and/or have the same length. During use, the needle 134 can be positioned at an insertion point (e.g., as shown in the illustration 115 of FIG. 1) where it can pierce the patient's skin. Once the needle 134 is in place, the needle 134 can be removed through the outer sleeve 136, and the distal portion 106 of the lead 102 can be placed through the lumen of the sleeve 136 and threaded under the skin. The sleeve 136 can then be optionally retracted outside the body of the patient. In other embodiments, the defibrillator system 100 can include a single introducer 132 that may be used to insert both leads 102, one after the other. In further embodiments, the defibrillator system 100 can include other suitable introducers or mechanisms for subcutaneously positioning the leads 102. For example, the defibrillator system 100 can include a scalpel or other type of cutting tool that can create a small incision (e.g., 1-5 mm) through which the distal portions 106 of the leads 102 can be threaded into the human body's subcutaneous space.

In various embodiments, the compartment 128 can define a sterile environment in which the leads 102, the introducers 132, and/or other components of the system 100 can be housed prior to use. In other embodiments, the leads 102 and/or portions thereof, the introducers 132, and/or other components can be sterilely packaged (e.g., in bags) and stored in the compartment 128. Even if the components are not sterilely packaged and/or a sterile environment is not maintained between unpacking the leads 102 and insertion of the electrodes 108, as may be the case in emergency situations, any resultant infection would typically be confined to small subcutaneous locations and easily treated. This type of infection is likely relatively minor when compared with the severity of the problem treated (i.e., cardiac arrest) and the types of systemic infections that can result from internal defibrillators attached directly to the heart and/or vasculature.

In further embodiments, the compartment 128 may be part of a housing or container that is separate from the pulse generator module 110. In this embodiment, a detachable container with the compartment 128 for housing the leads 102 may be releasably attached to the pulse generator module 110 using snaps, magnetic attraction, Velcro, interfacing surfaces, and/or other suitable connection means.

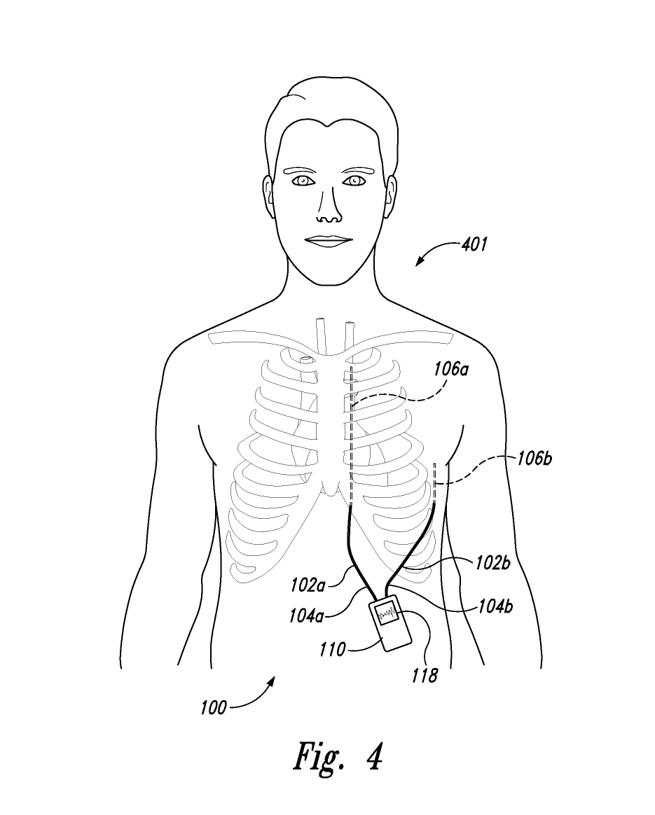

FIG. 4 is a partially schematic view illustrating the defibrillator system 100 described above with reference to FIGS. 1-3 implanted in a human patient 401 in accordance with an embodiment of the present technology. When the patient 401 experiences cardiac fibrillation, a person in the vicinity of the patient 401 carrying or having training with the defibrillator system 100 can use the system 100 to deliver one or more pulses of defibrillation energy to the patient 401. More specifically, the electrical leads 102 can be inserted subcutaneously into the patient 401 through a needle or cannula (e.g., the introducers 132 of FIG. 3), or through a small incision (e.g., 2 mm) in the skin. It has been shown that once the skin is penetrated, a moderately stiff lead can be threaded a considerable distance beneath the skin with little resistance. Accordingly, the leads 102 can be inserted through the patient's skin and the distal end portions 106 (including the electrodes 108 shown in FIGS. 1-3) can be threaded subcutaneously to predetermined target sites. For example, as shown in FIG. 4, the first lead 102a can be inserted proximate to the xiphoid process of the patient 401 and subcutaneously threaded at least 10 cm (e.g., 15 cm) such that the first electrode 108a (FIGS. 1-3) is positioned to the left side of the patient's upper sternum. The second lead 102b can be inserted proximate to the mid-axillary line of the patient and subcutaneously threaded at least 5 cm between intercostal spaces (e.g., the fifth and sixth intercostal spaces). The proximal portions 104 of the electrical leads 102 can be electrically coupled to the pulse generator module 110 (e.g., via the ports 112 shown in FIGS. 1 and 3) before or after the electrodes 108 (FIGS. 1-4) are positioned at their respective target sites. In other embodiments, the leads 102 may be pre-attached to the pulse generator module 110.

The pulse generator module 110 may be turned on (e.g., via the activation switch 122 shown in FIG. 1) and an indicator (e.g., the indicator light 124 of FIG. 1) can advise the user when the pulse generator module 110 is ready to deliver electrical shocks sufficient for cardiac defibrillation. The user can then manipulate the activation mechanism 126 (FIG. 1) on the pulse generator module 110 to initiate the delivery of defibrillation energy to the heart of the patient 401. In certain embodiments, electrodes (e.g., sense electrodes 108b and 108c shown in FIGS. 1-3) can detect the cardiac signals of the patient's heart, and this information can be processed via feedback algorithms 116 (FIG. 1) into QRS waves and/or other information related to the detected cardiac signals. The information can be provided to the user via the display 118 and used to guide the timing of the energy delivery. In other embodiments, the sensed cardiac data can further be used by control algorithms to indicate to the user when it is appropriate to apply defibrillation energy and/or automatically deliver defibrillation energy. The defibrillator system 100 may remain partially subcutaneously positioned as shown in FIG. 4 to provide further defibrillation until the patient reaches a hospital or other location for definitive care.

The partially subcutaneous defibrillator system 100 can deliver defibrillation energy to achieve internal cardiac defibrillation using subcutaneously positioned electrodes and an external electrical pulse generator. Because the shock provided by the defibrillator system 100 is delivered subcutaneously such that the impedance or resistance of the skin is bypassed, the defibrillator system 100 does not require the large electrodes or gel necessary for traditional external defibrillation. In addition, the subcutaneous placement of the electrodes 108 (FIGS. 1-3) reduces the level of electrical shock that is required for defibrillation. For example, the defibrillator system 100 may have a maximum energy output of about 80 J as compared to at least 175 J required for external defibrillation. The reduced size of the electrodes 108 and the energy required allows the defibrillator system 100 to be relatively small in size in comparison to external defibrillators. For example, in many embodiments the system 100 may be about the size of a smart phone. This allows users, such as physicians or other trained medical professionals, to regularly carry the defibrillator system 100 with them during daily activities making it readily available in the case of an emergency. For example, even though physicians routinely use a full-function external defibrillator in the hospital setting, external defibrillators are often not as readily available in hospitals when needed. In addition, the defibrillator system 100 can also be carried by medical professionals outside of the hospital and used in an emergency that may arise in the normal course of daily life, such as on the street, in the home, on the golf course, at a ballpark, while fishing, or in any setting remote from a hospital.

In addition, the defibrillator system 100 can be used by other, non-medical users trained to use the defibrillator system 100. For example, family members of persons who have heart rhythm abnormalities, irregular heartbeat, mild to moderate coronary artery disease, and/or other cardiac-related concerns may be trained to use the defibrillator system 100 and, therefore, be able to carry the defibrillator system 100 so that is readily available in case of emergency. Additionally, flight attendants can be trained to use the defibrillator system 100 for in-flight emergencies. Accordingly, the defibrillator system 100 can be used by trained individuals to save lives when medical personnel is not readily available with a cardiac defibrillator and cardio pulmonary resuscitation (“CPR”) is insufficient.

Moreover, VF and other cardiac fibrillation commonly occurs outside the hospital in previously healthy individuals having regular rhythm disturbances and those having electrical disturbances in the heart caused by reduced blood flow (ischemia) or myocardial infarction. In contrast to hospitalized patients, many persons experiencing out-of-hospital cardiac fibrillation have heart muscle that is substantially healthy, and therefore quick cardiopulmonary resuscitation and defibrillation can typically restore the individuals to normal active lives. Accordingly, the widespread availability of the portable defibrillator system 100 is expected to save many lives.

### Examples

The following Examples are illustrative of several embodiments of the present technology.

1. A portable external cardiac defibrillator system for treating a human patient, the cardiac defibrillator system comprising:
a first percutaneous lead having a proximal portion and a distal portion, wherein the distal portion of the first percutaneous lead comprises a first electrode, and wherein the distal portion of the first percutaneous lead is configured to be subcutaneously inserted into the human patient such that the first electrode is subcutaneously positioned proximate to an upper sternum of the human patient;
a second percutaneous lead having a proximal portion and a distal portion, wherein the distal portion of the second electrical lead comprises a second electrode, and wherein the distal portion of the second percutaneous lead is configured to be subcutaneously inserted into the human patient such that the second electrode is positioned between intercostal spaces of the human patient; and
an external portable pulse generator module configured to be operably coupled to the first and second electrical leads and positioned external to the human patient, wherein the pulse generator module is configured to deliver therapeutic doses of electrical energy to a heart of the human patient via the first and second electrical leads.

2. The portable external cardiac defibrillator system of example 1 wherein the pulse generator module is configured to deliver electrical energy of at most 80 J.

3. The portable external cardiac defibrillator system of example 1 or 2 wherein the distal portion of the first percutaneous lead is configured to be inserted into the human patient at a xiphoid process of the human patient and delivered to the upper sternum.

4. The portable external cardiac defibrillator system of any one of examples 1-3 wherein the first electrode comprises a defibrillation coil and a sensing portion, the defibrillation coil having a length of 5-10 cm.

5. The portable external cardiac defibrillator system of any one of examples 1-4 wherein 10-20 cm of the first percutaneous lead is configured to be inserted subcutaneously into the human patient, and wherein 5-10 cm of the second percutaneous lead is configured to be inserted subcutaneously into the human patient.

6. The portable external cardiac defibrillator system of any one of examples 1-5 wherein the first percutaneous lead has a length of about 20-30 cm, and wherein the second percutaneous lead has a length of about 10-20 cm.

7. The portable external cardiac defibrillator system of any one of examples 1-6, further comprising an introducer having a sleeve and a needle within the sleeve, wherein the needle is configured to form an opening in subcutaneous tissue of the human patient, and wherein the distal portion of the first percutaneous lead is configured to be threaded through the sleeve into the opening to a subcutaneous target site.

8. The portable external cardiac defibrillator system of any one of examples 1-7 wherein:

the first percutaneous lead is a first color;
the second percutaneous lead is a second color different from the first color; and
the pulse generator module further comprises a first port having the first color and configured to receive the first percutaneous lead, and a second port having the second color and configured to receive the second percutaneous lead.

9. The portable external cardiac defibrillator system of any one of examples 1-8 wherein:

the distal portion of the first percutaneous lead comprises a third electrode electrically isolated from the first electrode; and
the second and third electrodes are configured to detect cardiac activity.

10. The portable external cardiac defibrillator system of example 9 wherein the pulse generator module further comprises a display, and wherein the monitor is configured to display electrocardiogram recordings detected by the first and second sensing portions.

11. The portable external cardiac defibrillator system of any one of examples 1-10 wherein the pulse generator module comprises an indicator light, and wherein the indicator light illuminates when the pulse generator module is sufficiently charged for defibrillation.

12. The portable external cardiac defibrillator system of any one of examples 1-11 wherein the pulse generator module comprises a first activation mechanism and a second activation button, and wherein the first and second activations buttons must both be manipulated by a user to administer the therapeutic doses of electrical energy via the first and second percutaneous leads.

13. The portable external cardiac defibrillator system of any one of examples 1-12 wherein the pulse generator module comprises a compartment, and wherein the first and second electrodes are sterilely packaged within the compartment.

14. The portable external cardiac defibrillator system of any one of examples 1-13, further comprising:
a housing configured to be worn externally by a user,
wherein the pulse generator module is contained in the housing, and
wherein the proximal portions of the first and second percutaneous leads are coupled to the pulse generator module in the housing.

15. The portable external cardiac defibrillator system of any one of examples 1-14, further comprising a compartment sized to house the first and second percutaneous leads, wherein the compartment is detachably connected to the pulse generator module.

16. A portable external cardiac defibrillator system for treating a human patient, the cardiac defibrillator system comprising:
a first electrode;
a second electrode, wherein the first and second electrodes are configured to be external to the human patient and implanted subcutaneously in the human patient during a cardiac emergency; and
an electrical pulse generator external to the human patient and operably coupled to the first and second electrodes, wherein the electrical pulse generator is configured to deliver an electrical shock to the human patient via the first and second electrodes while the first and second electrodes are subcutaneously implanted to provide internal defibrillation to the human patient.

17. The portable external cardiac defibrillator system of 16 wherein the electrical pulse generator is configured to be manually activated by a user to deliver the electrical shock.

18. A portable external cardiac defibrillator system, comprising:
a portable case configured to be carried externally by a user;
a first lead coupled to the case and having a first active end portion, wherein the first active end portion is configured to be implanted at a first subcutaneous position in a human patient;
a second lead coupled to the case and having a second active end portion, wherein the second active end portion is configured to be implanted at a second subcutaneous position in the human patient, wherein the first and second subcutaneous positions are proximate to a heart of the human patient; and
a pulse generator module housed within the case, wherein the pulse generator is configured to be electrically coupled to the first and second electrical leads and positioned external to the human patient, and wherein the first and second leads are configured to deliver a therapeutic dose of electrical energy to the heart of the human patient to achieve internal defibrillation.

19. The portable external cardiac defibrillator system of example 18 wherein:
the first active end portion comprises a defibrillation coil and a first sense electrode; and
the second active end portion comprises a second sense electrode.

20. The portable external cardiac defibrillator system of example 18 or 19 wherein the first and second active end portions are configured to detect electrocardiogram signals from the heart of the human patient.

21. The portable external cardiac defibrillator system of example 20, further comprising a display operably coupled to the first and second leads, wherein the display is configured to illustrate electrocardiogram recordings detected by the first and second active end portions to the user.

22. The portable external cardiac defibrillator system of any one of examples 18-21 wherein the pulse generator module comprises an indicator configured to indicate to a user when capacitors of the pulse generator module are charged.

23. The cardiac defibrillator system of any one of examples 18-22 wherein the case comprises a sterile compartment, wherein the sterile compartment is configured to house the first and second leads.

24. A method of defibrillating a human patient, comprising:
positioning a distal portion of a first lead subcutaneously at a first target site proximate to an upper sternum of the human patient;
positioning a distal portion of a second lead subcutaneously at a second target site between intercostal spaces of the human patient; and
delivering a therapeutic dose of electrical energy to a heart of the human patient with a portable pulse generator operably coupled to the first and second leads, wherein the pulse generator is connected to the first and second leads external to the human patient.

25. The method of example 24 wherein positioning the distal portions of the first and second leads subcutaneously further comprises:
forming a first small incision through subcutaneous tissue of the human patient proximate to a xiphoid process of the human patient;
subcutaneously threading the distal portion of the first lead to the first target site;
forming a second small incision through subcutaneous tissue of the human patient proximate to the mid-axillary line of the human patient; and
subcutaneously threading the distal portion of the second lead to the second target site between a fifth intercostal space and a sixth intercostal space of the human patient.

26. The method of example 24 or 25 wherein positioning the distal portions of the first and second leads subcutaneously further comprises:
inserting a first introducer through subcutaneous tissue of the human patient proximate to a xiphoid process of the human patient;
threading the distal portion of the first lead through the introducer to the first target site;
inserting a second introducer subcutaneous tissue of the human patient proximate to the mid-axillary line of the human patient; and
threading the distal portion of the second lead through the introducer to the second target site.

27. The method of any one of examples 24-26 wherein delivering the therapeutic dose of electrical energy to the heart of the human patient with the pulse generator comprises delivering an electrical pulse of at most 80 J.

28. The method of any one of examples 24-27 wherein:
positioning the distal portion of the first lead subcutaneously at the first target site comprises subcutaneously threading about 10-20 cm of the first lead to the first target site; and
positioning the distal portion of the second lead subcutaneously at the first target site comprises subcutaneously threading about 5-10 cm of the second lead to the second target site.

29. The method of any one of examples 24-28, further comprising:
positioning a proximal portion of the first lead in a first port of the pulse generator, wherein the first lead and the first port have a first color; and
positioning a proximal portion of the second lead in a second port of the pulse generator, wherein the second lead and the second port have a second color different from the first color.

30. The method of any one of examples 24-29, further comprising:
recording electrocardiograms via sense electrodes at the distal portions of the first and second leads; and
displaying the electrocardiograms on the pulse generator.

31. The method of any one of examples 24-30, further comprising:
recording electrocardiograms via sense electrodes at the distal portions of the first and second leads; and
automatically applying the therapeutic doses of electrical energy to the heart of the human patient in response to data from the electrocardiograms.

32. The method of any one of examples 24-31, further comprising:
recording electrocardiograms via sense electrodes at the distal portions of the first and second leads; and
indicating to a user when to administer the therapeutic dose of electrical energy based on the recorded electrocardiograms.

33. The method of any one of examples 24-32, further comprising indicating on the pulse generator when capacitors in the pulse generator is fully charged.

34. The method of any one of examples 24-33 wherein delivering the therapeutic dose of electrical energy to the heart of the human patient with the pulse generator comprises simultaneously manually manipulating a first activation member and a second activation member to administer the therapeutic dose of electrical energy.

35. The method of any one of examples 24-34 wherein the pulse generator is housed in a case, wherein the case is configured to be worn externally by a user, and wherein the first and second leads each have a proximal portion coupled to the case.

36. The method of any one of examples 24-35, further comprising:
maintaining the distal portions of the first and second leads at the first and second target sites after an initial defibrillation; and
delivering subsequent therapeutic doses of electrical energy to the heart of the human patient via the first and second leads.

### CONCLUSION

From the foregoing, it will be appreciated that specific embodiments of the technology have been described herein for purposes of illustration, but that various modifications may be made without deviating from the disclosure. Certain aspects of the new technology described in the context of particular embodiments may be combined or eliminated in other embodiments. Additionally, although advantages associated with certain embodiments of the new technology have been described in the context of those embodiments, other embodiments may also exhibit such advantages and not all embodiments need necessarily exhibit such advantages to fall within the scope of the technology. Accordingly, the disclosure and associated technology can encompass other embodiments not expressly shown or described herein.

I/we claim:

1. A portable external cardiac defibrillator system for treating a human patient, the cardiac defibrillator system comprising:
a first percutaneous lead having a proximal portion and a distal portion, wherein the distal portion of the first percutaneous lead comprises a first electrode, and wherein the distal portion of the first percutaneous lead is configured to be subcutaneously inserted into the human patient such that the first electrode is subcutaneously positioned proximate to an upper sternum of the human patient;
a second percutaneous lead having a proximal portion and a distal portion, wherein the distal portion of the second electrical lead comprises a second electrode, and wherein the distal portion of the second percutaneous lead is configured to be subcutaneously inserted into the human patient such that the second electrode is positioned between intercostal spaces of the human patient; and
an external portable pulse generator module configured to be operably coupled to the first and second electrical leads and positioned external to the human patient, wherein the pulse generator module is configured to deliver therapeutic doses of electrical energy to a heart of the human patient via the first and second electrical leads.

2. The portable external cardiac defibrillator system of claim 1 wherein the pulse generator module is configured to deliver electrical energy of at most 80 J.

3. The portable external cardiac defibrillator system of claim 1 wherein the distal portion of the first percutaneous lead is configured to be inserted into the human patient at a xiphoid process of the human patient and delivered to the upper sternum.

4. The portable external cardiac defibrillator system of claim 1 wherein the first electrode comprises a defibrillation coil and a sensing portion, the defibrillation coil having a length of 5-10 cm.

5. The portable external cardiac defibrillator system of claim 1 wherein 10-20 cm of the first percutaneous lead is configured to be inserted subcutaneously into the human patient, and wherein 5-10 cm of the second percutaneous lead is configured to be inserted subcutaneously into the human patient.

6. The portable external cardiac defibrillator system of claim 1 wherein the first percutaneous lead has a length of about 20-30 cm, and wherein the second percutaneous lead has a length of about 10-20 cm.

7. The portable external cardiac defibrillator system of claim 1, further comprising an introducer having a sleeve and a needle within the sleeve, wherein the needle is configured to form an opening in subcutaneous tissue of the human patient, and wherein the distal portion of the first percutaneous lead is configured to be threaded through the sleeve into the opening to a subcutaneous target site.

8. The portable external cardiac defibrillator system of claim 1 wherein:
the first percutaneous lead is a first color;
the second percutaneous lead is a second color different from the first color; and
the pulse generator module further comprises a first port having the first color and configured to receive the first percutaneous lead, and a second port having the second color and configured to receive the second percutaneous lead.

9. The portable external cardiac defibrillator system of claim 1 wherein:
the distal portion of the first percutaneous lead comprises a third electrode electrically isolated from the first electrode; and
the second and third electrodes are configured to detect cardiac activity.

10. The portable external cardiac defibrillator system of claim 9 wherein the pulse generator module further comprises a display, and wherein the display is configured to display electrocardiogram recordings detected by the first and second sensing portions.

11. The portable external cardiac defibrillator system of claim 1 wherein the pulse generator module comprises an indicator light, and wherein the indicator light illuminates when the pulse generator module is sufficiently charged for defibrillation.

12. The portable external cardiac defibrillator system of claim 1 wherein the pulse generator module comprises a first activation mechanism and a second activation mechanism, and wherein the first and second activations buttons must both be manipulated by a user to administer the therapeutic doses of electrical energy via the first and second percutaneous leads.

13. The portable external cardiac defibrillator system of claim 1 wherein the pulse generator module comprises a compartment, and wherein the first and second electrodes are sterilely packaged within the compartment.

14. The portable external cardiac defibrillator system of claim 1, further comprising:
a housing configured to be worn externally by a user,
wherein the pulse generator module is contained in the housing, and
wherein the proximal portions of the first and second percutaneous leads are coupled to the pulse generator module in the housing.

15. The portable external cardiac defibrillator system of claim 1, further comprising a compartment sized to house the first and second percutaneous leads, wherein the compartment is detachably connected to the pulse generator module.

16. A portable external cardiac defibrillator system for treating a human patient, the cardiac defibrillator system comprising:
a first electrode;
a second electrode, wherein the first and second electrodes are configured to be external to the human patient and implanted subcutaneously in the human patient during a cardiac emergency; and
an electrical pulse generator external to the human patient and operably coupled to the first and second electrodes, wherein the electrical pulse generator is configured to deliver an electrical shock to the human patient via the first and second electrodes while the first and second electrodes are subcutaneously implanted to provide internal defibrillation to the human patient.

17. The portable external cardiac defibrillator system of 16 wherein the electrical pulse generator is configured to be manually activated by a user to deliver the electrical shock.

18. A portable external cardiac defibrillator system, comprising:
a portable case configured to be carried externally by a user;
a first lead coupled to the case and having a first active end portion, wherein the first active end portion is configured to be implanted at a first subcutaneous position in a human patient;
a second lead coupled to the case and having a second active end portion, wherein the second active end portion is configured to be implanted at a second subcutaneous position in the human patient, wherein the first and second subcutaneous positions are proximate to a heart of the human patient; and
a pulse generator module housed within the case, wherein the pulse generator is configured to be electrically coupled to the first and second electrical leads and positioned external to the human patient, and wherein the first and second leads are configured to deliver a therapeutic dose of electrical energy to the heart of the human patient to achieve internal defibrillation.

19. The portable external cardiac defibrillator system of claim 18 wherein:
the first active end portion comprises a defibrillation coil and a first sense electrode; and
the second active end portion comprises a second sense electrode.

20. The portable external cardiac defibrillator system of claim 18 wherein the first and second active end portions are configured to detect electrocardiogram signals from the heart of the human patient.

21. The portable external cardiac defibrillator system of claim 20, further comprising a display operably coupled to the first and second leads, wherein the display is configured to illustrate electrocardiogram recordings detected by the first and second active end portions to the user.

22. The portable external cardiac defibrillator system of claim 18 wherein the pulse generator module comprises an indicator configured to indicate to a user when capacitors of the pulse generator module are charged.

23. The cardiac defibrillator system of claim 18 wherein the case comprises a sterile compartment, wherein the sterile compartment is configured to house the first and second leads.

24. A method of defibrillating a human patient, comprising:
positioning a distal portion of a first lead subcutaneously at a first target site proximate to an upper sternum of the human patient;
positioning a distal portion of a second lead subcutaneously at a second target site between intercostal spaces of the human patient; and
delivering a therapeutic dose of electrical energy to a heart of the human patient with a portable pulse generator operably coupled to the first and second leads, wherein the pulse generator is connected to the first and second leads external to the human patient.

25. The method of claim 24 wherein positioning the distal portions of the first and second leads subcutaneously further comprises:
forming a first small incision through subcutaneous tissue of the human patient proximate to a xiphoid process of the human patient;
subcutaneously threading the distal portion of the first lead to the first target site;
forming a second small incision through subcutaneous tissue of the human patient proximate to the mid-axillary line of the human patient; and
subcutaneously threading the distal portion of the second lead to the second target site between a fifth intercostal space and a sixth intercostal space of the human patient.

26. The method of claim 24 wherein positioning the distal portions of the first and second leads subcutaneously further comprises:
inserting a first introducer through subcutaneous tissue of the human patient proximate to a xiphoid process of the human patient;
threading the distal portion of the first lead through the introducer to the first target site;
inserting a second introducer subcutaneous tissue of the human patient proximate to the mid-axillary line of the human patient; and
threading the distal portion of the second lead through the introducer to the second target site.

27. The method of claim 24 wherein delivering the therapeutic dose of electrical energy to the heart of the human patient with the pulse generator comprises delivering an electrical pulse of at most 80 J.

28. The method of claim 24 wherein:
positioning the distal portion of the first lead subcutaneously at the first target site comprises subcutaneously threading about 10-20 cm of the first lead to the first target site; and
positioning the distal portion of the second lead subcutaneously at the first target site comprises subcutaneously threading about 5-10 cm of the second lead to the second target site.

29. The method of claim 24, further comprising:
positioning a proximal portion of the first lead in a first port of the pulse generator, wherein the first lead and the first port have a first color; and
positioning a proximal portion of the second lead in a second port of the pulse generator, wherein the second lead and the second port have a second color different from the first color.

30. The method of claim 24, further comprising:
recording electrocardiograms via sense electrodes at the distal portions of the first and second leads; and
displaying the electrocardiograms on the pulse generator.

31. The method of claim 24, further comprising:
recording electrocardiograms via sense electrodes at the distal portions of the first and second leads; and
automatically applying the therapeutic doses of electrical energy to the heart of the human patient in response to data from the electrocardiograms.

32. The method of claim 24, further comprising:
recording electrocardiograms via sense electrodes at the distal portions of the first and second leads; and
indicating to a user when to administer the therapeutic dose of electrical energy based on the recorded electrocardiograms.

33. The method of claim 24, further comprising indicating on the pulse generator when capacitors in the pulse generator is fully charged.

34. The method of claim 24 wherein delivering the therapeutic dose of electrical energy to the heart of the human patient with the pulse generator comprises simultaneously manually manipulating a first activation member and a second activation member to administer the therapeutic dose of electrical energy.

35. The method of claim 24 wherein the pulse generator is housed in a case, wherein the case is configured to be worn externally by a user, and wherein the first and second leads each have a proximal portion coupled to the case.

36. The method of claim 24, further comprising:
maintaining the distal portions of the first and second leads at the first and second target sites after an initial defibrillation; and
delivering subsequent therapeutic doses of electrical energy to the heart of the human patient via the first and second leads.
